(19) United States
(12) Patent Application Publication
(10) Pub. No.: US 2011/0059540 A1
(43) Pub. Date: Mar. 10, 2011

(54) IDENTIFYING SUSCEPTIBILITY OF A SUBJECT TO CARDIAC THERAPY BASED ON DETERMINATION OF A CARDIAC TROPONIN, SCD40L, AND C-REACTIVE PROTEIN

(76) Inventors: Georg Hess, Mainz (DE); Andrea Horsch, Mannheim (DE); Dietmar Zdunek, Tutzing (DE)

(21) Appl. No.: 12/944,833

(22) Filed: Nov. 12, 2010

Related U.S. Application Data
(63) Continuation of application No. PCT/EP2009/056646, filed on May 29, 2009.

(30) Foreign Application Priority Data
May 29, 2008 (EP) 08157148.1

(51) Int. Cl. G01N 33/53; G01N 33/00; B01L 3/00

(52) U.S. Cl. 436/86; 422/63; 422/430

(57) ABSTRACT

Disclosed is a method for identifying a subject being susceptible to a cardiac therapy based on determination of a cardiac troponin T and the additional determination of C-reactive protein (CRP) or sCD40L (soluble CD40 ligand) in a sample of a subject with stable coronary heart disease and a history of an acute cardiovascular event. Also disclosed is a method for predicting the risk of mortality and/or a further acute cardiovascular event for a subject with stable coronary heart disease and a history of acute cardiovascular event based on the determination of the aforementioned markers. Further disclosed are kits and devices adapted to carry out the disclosed methods.

### RELATED APPLICATIONS

This application is a continuation of PCT/EP2009/056646 filed May 29, 2009 and claims priority to EP 08157148.1 filed May 29, 2008.

### FIELD OF THE INVENTION

The present invention relates to a method for identifying a subject being susceptible to a cardiac therapy based on determination of a cardiac troponin T and the additional determination of C-reactive protein (CRP) or soluble CD40 ligand (sCD40L) in a sample of a subject with stable coronary heart disease and a history of an acute cardiovascular event. Moreover, the present invention relates to a method for predicting the risk of mortality and/or a further an acute cardiovascular event for a subject with stable coronary heart disease and a history of acute cardiovascular event based on the determination of the aforementioned markers. Further encompassed by the present invention are kits and devices adapted to carry out the methods of the present invention.

### BACKGROUND OF THE INVENTION

An aim of modern medicine is to provide personalized or individualized treatment regimens. Those are treatment regimens which take into account a patient's individual needs or risks. A particularly important risk is the presence of a stable coronary heart disease, particularly in patient which have a history of an acute cardiovascular event such as an acute stable coronary syndrome. Stable coronary heart diseases belong to the leading cause of morbidity and mortality in the Western hemisphere.

Stable coronary heart disease (abbreviated CHD) is the result of the accumulation of atheromatous plaques within the walls of the arteries that supply the myocardium (the muscle of the heart). While the symptoms and signs of stable coronary heart disease are noted in the advanced state of disease, most individuals with stable coronary heart disease show no evidence of disease for decades as the disease progresses before the first onset of symptoms, often a “sudden” heart attack, finally arise. After decades of progression, some of these atheromatous plaques may rupture and (along with the activation of the blood clotting system) start limiting blood flow to the heart muscle.

Several risk factors leading to various coronary heart disorders are known. One example is the level of total and of HDL cholesterol in the blood. However, the predictive value of a cholesterol level is low, and the identification of a large number of individuals having a high coronary heart event risk profile is not possible by measuring the cholesterol level.

In order to diagnose coronary heart disease a variety of markers has been proposed, e.g., high sensitive C-reactive protein (hsCRP); placental growth factor (PIGF), soluble CD40 Ligand (sCD40L), IL-10, ICAM-1, VCAM-1, E-selectin, P-selectin, IL-6, VEGF, Fibrinogen, soluble fibrin, anti-oxLDL, MCP-1, procoagulant tissue factor (TF), or von Willebrand Factor (vWF). Some of the aforementioned markers are known and characterized, and their role in the pathophysiology of coronary diseases is established, contrary to others which have not been examined in detail in respect to their prognostic value.

The use of cardiac troponins is well established for the diagnosis of acute cardiovascular events (see e.g., EP1890153 A1). Recently, it was proposed that cardiac troponins are suitable for predicting the risk of developing coronary heart disease. Results obtained by Zethelius et al. (Circulation 2006, 113: 1071-1078) showed that a cardiac troponin could predict death and coronary heart disease in 70 years old men. Moreover, there is strong evidence that very low plasma concentrations of troponin Tin patients with stable chronic heart failure are of prognostic value regarding adverse outcomes (Latini et al., Circulation 2007, 116: 1242 to 1249, Val-HeFT study).

Even though the diagnostic value of several markers is established, and some have been found to give highly important and exact diagnostic information relative to a certain coronary heart event/disease, the use of marker compositions may be of higher prognostic value. The use of a combination of markers for coronary heart disease, however, has only restrictedly been described. However, combinations of marker giving very precise diagnostic information or which can be used to establish a coronary heart risk profile of a patient are highly desired.

Thus, the conventional diagnostic techniques, specifically for patients suffering from stable coronary heart disease, may not allow for a reliable prognosis and/or risk assessment. Thus, a personalized treatment regimen or the risk of mortality and/or a cardiovascular event may not be determined with sufficient accuracy.

Therefore, there is a need for a reliable general risk stratification including the risk for mortality or cardiovascular events in patients suffering from a stable coronary heart disease. Moreover, there is a need for diagnostic or prognostic measures which allow individual risk stratification for a patient who is suspected to be in need for a certain treatment regimen.

The technical problem underlying the present invention can be seen as the provision of means and methods for complying with the aforementioned needs.

The technical problem is solved by the embodiments characterized in the claims and herein below.

### SUMMARY OF THE INVENTION

Accordingly, the present invention relates to a method for identifying a subject being susceptible to a cardiac therapy, said subject suffering from stable coronary heart disease and a history of at least one acute cardiovascular event comprising the steps of
a) determining the amount of a cardiac troponin in a sample of said subject,
b) comparing the amount of a cardiac troponin determined in step a) to a reference amount for a cardiac troponin, and
   b1) if the determined amount of said cardiac troponin is larger than the reference amount for a cardiac troponin, determining the amount of C-reactive protein (CRP) in a sample of said subject, and comparing the, thus, determined amount of CRP to a reference amount for CRP, or
   b2) if the determined amount of said cardiac troponin is lower than the reference amount for a cardiac troponin, determining the amount of sCD40L (soluble CD40L ligand) in a sample of said subject, and comparing the, thus, determined amount of sCD40L to a reference amount for sCD40L, and
c) identifying a subject being susceptible to a cardiac therapy.

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and subclassification of a subject in need of a cardiac therapy. The method may be carried out manually or assisted by automation. Preferably, step (a) and/or (b) may in total or in part be assisted by automation, e.g., by a suitable robotic and sensory equipment for the determination in steps (a), (b1) and/or (b2) or a computer-implemented comparisons in step (b), (b1) and/or (b2).

### BRIEF DESCRIPTION OF THE FIGURES

FIG. 1: Survival by sCD40L and troponin T (below and above median) in individuals with stable coronary heart disease and a history of unstable angina: The determination of sCD40L in patients with troponin T levels below the median adds further prognostic information (see Examples).

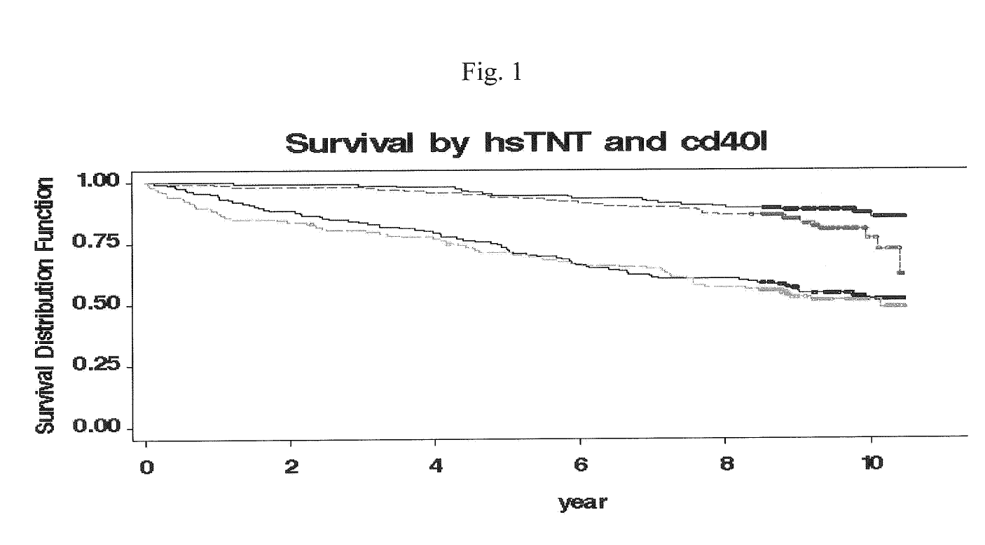

FIG. 2: Survival by CRP and troponin T (below and above median) in individuals with stable coronary heart disease and a history of unstable angina: The determination of CRP in patients with troponin T levels above the median adds further prognostic information (see Examples).

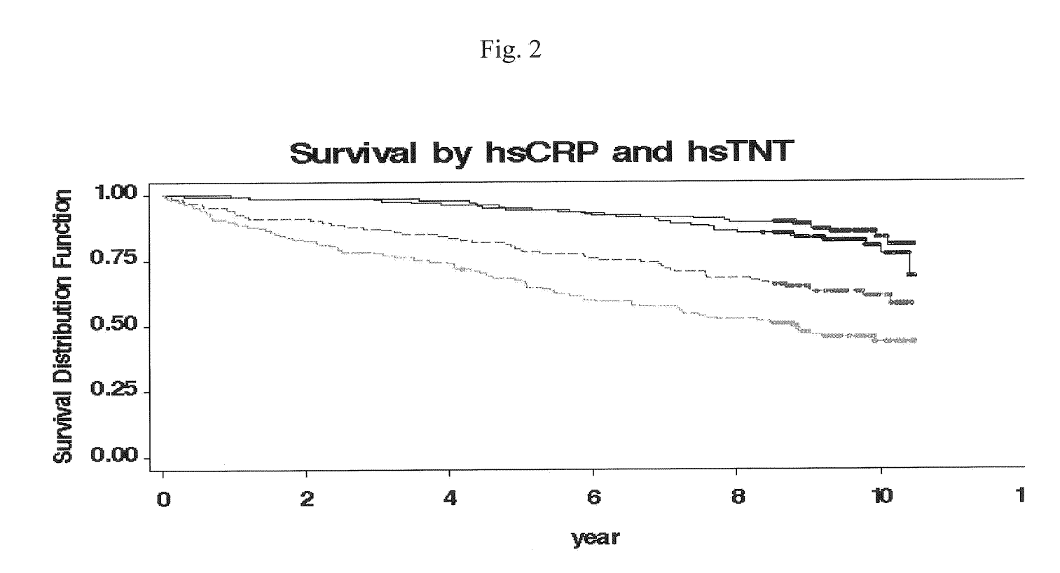

### DETAILED DESCRIPTION OF THE INVENTION

The term “identifying” as used herein means assessing whether a subject will be susceptible for a cardiac therapy or not, and, thus, requires a cardiac therapy or not. Moreover, for a subject who is susceptible to a cardiac therapy the advantages of said therapy shall outweigh the disadvantages (particularly disadvantages caused by adverse side effect of a certain treatment regimen, but also with respect to the costs). Also, for a subject who is not susceptible to a cardiac therapy, the disadvantages (particularly, with respect to adverse side effects but also with respect to the costs due to an over-treatment) of said therapy will outweigh the advantages. Particularly, if a subject does not require a certain cardiac therapy, costs that would result from on over-treatment will be saved and/or adverse side effects can be avoided if said subject is not put on a certain cardiac therapy.

As will be understood by those skilled in the art, such an assessment (whether a subject is susceptible or not to a cardiac therapy) is usually not intended to be correct for all (i.e., 100%) of the subjects to be identified. The term, however, requires that a statistically significant portion of subjects can be identified (e.g., a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly identified by the method of the present invention.

The term “subject” as used herein relates to animals, preferably mammals, and, more preferably, humans. However, it is envisaged by the method of the present invention that the subject shall be a subject exhibiting stable coronary heart disease and, and, therefore, preferably shall be in need for a cardiac therapy. The term “coronary heart disease”, abbreviated CHD, also called coronary artery disease (CAD) or atherosclerotic heart disease, is known to the person skilled in the art. Preferably, the term coronary heart disease (CHD) refers to a condition in which small blood vessels that supply blood and oxygen to the heart are narrowed. Coronary heart disease is usually caused by a condition called atherosclerosis, which occurs when plaque builds up on the walls of the arteries. This causes a narrowing of the blood vessels. Particularly, CHD is the result of the accumulation of atheromatous plaques within the walls of the arteries that supply the myocardium (the muscle of the heart). Preferably, a subject with stable coronary heart disease has at least 50% stenosis (and, thus at least 50% occlusion), in at least one major coronary artery. How to assess the degree of occlusion of a coronary artery is well known in the art, preferably, the degree is assessed by coronary angiography. While the symptoms and signs of coronary heart disease are noted in the advanced state of disease, most individuals with coronary heart disease show no evidence of disease for decades as the disease progresses before the first onset of symptoms of an acute event, often a “sudden” heart attack, finally arise.

The term “stable coronary heart disease”, preferably, does not include acute cardiovascular syndromes. Particularly, said term does not include STEMI (ST-elevation myocardial infarction); NSTEMI (non ST-elevation myocardial infarction) and unstable angina pectoris (but, preferably, does include stable angina pectoris). Preferably, the subject shall have a cardiac troponin level, preferably, a troponin T level lower than, 0.25 ng/ml, more preferably, lower than 0.05 ng/ml, and, most preferably, lower than 0.1 ng/ml. The term “cardiac troponin level” as used herein relates to the concentration of a cardiac troponin, preferably of TnT, preferably, in a blood, blood plasma or blood serum sample of a subject as defined herein.

However, the subject shall, preferably, have a history of events belonging to the acute cardiovascular syndrome, i.e., the subject shall have exhibited at least one acute cardiovascular event in the past. Acute cardiovascular events are, preferably, acute coronary syndromes (ACS). ACS patients can show unstable angina pectoris (UAP) or myocardial infarction (MI). MI can be an ST-elevation MI (STEMI) or a non-ST-elevated MI (NSTEMI). The occurring of an ACS can be followed by a left ventricular dysfunction (LVD) and symptoms of heart failure. How to diagnose an acute cardiovascular event is well known in the art.

Although the subject shall have history at least one acute cardiovascular event, it is particularly contemplated that said subject shall not have exhibited an acute cardiovascular event recently, preferably not within one week, three month, one year, or more preferably six month, or even more preferably one month prior to carrying out the method of the present invention. Accordingly, the at least one acute cardiovascular event has, preferably, occurred more than, one week, one month, three month, six month or one year prior to determining the various markers as specified herein (more precisely: prior to obtaining the sample to be analyzed). It is, particularly, contemplated that an acute cardiovascular event did not occur within one month prior to carrying out the method of the present invention.

The term “cardiac therapy” encompasses, preferably, those treatment regimens which are suitable for the treatment of coronary heart disease and, particularly, of stable coronary heart disease. Preferably, said treatment regimens aim to treat the symptoms and signs of coronary heart disease and aim prevent a further progression of coronary heart disease. However, the term “cardiac therapy”, preferably, may also encompass anti-inflammation and anti-platelet treatment regimens.

Preferably, said treatment regimens are selected from interventional therapies (and thus invasive methods of treatment) and drug-based therapies. Drugs suitable for the treatment of coronary heart disease are known in the art. Preferably, said drugs are selected from the group consisting of ACE (angiotensin converting enzyme)-inhibitors, Angiotensin receptor blockers, beta-adrenergic blockers, combined alpha and beta blockers, calcium antagonists (calcium channel blockers), Glycoprotein IIb/IIIa inhibitors, Heparin, macumar, low molecular weight heparins, nitrates, oral anticoagulants (e.g., warfarin), lipid lowering drugs, and thrombocyte aggregation inhibitors (and, thus, platelet inhibitors). Also combinations of the aforementioned pharmaceuticals can be administered.

ACE-inhibitors are known to the person skilled in the art. Examples include benazepril, captopril, cilazapril, enalapril, fosinopril, lisinopril, moexipril, perindopril, quinapril, ramipril, spirapril, and trandolapril.

β-adrenergic blockers (non-selective and β1-selective) are known to the person skilled in the art. Examples include acebutolol, alprenolol, atenolol, betaxolol, bisoprolol, bupranolol, carazolol, carteolol, carvedilol, celiprolol, metipranolol, metoprolol, nadolol, nebivolol, oxprenolol, penbutolol, pindolol, propanolol, sotalol, tanilolol, and timolol.

The term “aldosterone antagonist” relates to a compound being capable of counteracting the effect of aldosterone, e.g., by competitive blockage of aldosterone receptors found in renal tubules. Preferably, said aldosterone antagonist is spironolacton or eplerenone

Lipid-lowering drugs are known to the person skilled in the art. Examples include fibrates (e.g., bezofibrate, clofibrate, etofibrate, etophylline clofibrate, fenofibrate, gemfibrozil), nicotinic acid and analogs thereof (e.g., nicotinic acid, acipimox), statins (e.g., simvastatin, lovastatin, pravastatin, fluvastatin, atorvastatin, cerivastatin), anion exchanger resins (e.g., colestyramine, colestipol), probucol, and sitosterol. Preferred lipid-lowering drugs in the present context are statins (which are also suitable for anti-inflammation therapy).

Inhibitors or antagonists of the platelet glycoprotein IIb/IIIa receptor are known to the person skilled in the art. Examples include monoclonal or polyclonal antibodies, tirofiban, eptifibatide, and the like. In a preferred embodiment of the present invention, the glycoprotein IIb/IIIa receptor inhibitor is an antibody, in particular the antibody known under the name abciximab. Abciximab is a Fab fragment antibody which is available under the name ReoPro from Centocor Europe BV.

Thrombocyte aggregation inhibitors are known to the person skilled in the art (frequently also referred to as platelet inhibitors) and include any drugs capable of inhibiting the aggregation of thrombocytes (platelets). Examples are inhibitors of cyclooxygenase, particularly COX-1 (particularly acetylsalicylic acid); ADP inhibitors (which inhibit binding of adenosine phosphate to its receptors on thrombocyates, e.g., ticlopidin or clopidogrel); inhibitors or antagonists of the platelet glycoprotein IIb/IIIa receptor (see above); dipyridamol; sulfinpyrazone; macumar, heparin, dextran 40. For platelet therapy using platelet inhibitors, see also Braunwald's Heart Disease: A Textbook of Cardiovascular Medicine, Publisher: Saunders, 8th edition, 2007, chapter 80 Hemostasis, thrombosis, fibrinolysis and cardiovascular disease.

Invasive methods of treatment include intervention by surgery, microsurgery or other therapies affecting the cardiovascular system and, preferably, the heart. This is, preferably, achieved by enhancing the blood flow throughout the blood vessels supporting the heart, i.e., the coronary blood vessels. Those blood vessels may be impaired due to, e.g., thrombotic or atherosclerotic plaques. Accordingly, invasive methods shall, preferably, comprise a destruction and/or removal of such plaques and a restoration of the vessel, if necessary. Preferred invasive methods in accordance with the present invention are selected from the group consisting of percutaneous coronary angioplasty, percutaneous transluminal coronary balloon angioplasty, laser angioplasty, coronary stent implantation, bypass implantation or intraluminal techniques aiming to restore blood flow, vessel patency, stabilize plaque, and/or reduce intracoronary thrombus load. Invasive methods are particularly contemplated for subjects which are highly susceptible for a cardiac therapy.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

The term “cardiac troponin” refers to all troponin isoforms expressed in cells of the heart and, preferably, the subendocardial cells. Cardiac troponins are known to be a marker for necrosis of cardiac tissue. These isoforms are well characterized in the art as described, e.g., in Anderson 1995, Circulation Research, vol. 76, no. 4: 681-686 and Ferrieres 1998, Clinical Chemistry, 44: 487-493. Preferably, cardiac troponin refers to troponin T and/or troponin I, and, most preferably, to troponin T. It is to be understood that isoforms of troponins may be determined in the method of the present invention together, i.e., simultaneously or sequentially, or individually, i.e., without determining the other isoform at all. Amino acid sequences for human troponin T and human troponin I are disclosed in Anderson, loc cit and Ferrieres 1998, Clinical Chemistry, 44: 487-493.

The term “cardiac troponin” encompasses also variants of the aforementioned specific troponins, i.e., preferably, of troponin I, and more preferably, of troponin T. Such variants have at least the same essential biological and immunological properties as the specific cardiac troponins. In particular, they share the same essential biological and immunological properties if they are detectable by the same specific assays referred to in this specification, e.g., by ELISA assays using polyclonal or monoclonal antibodies specifically recognizing the cardiac troponins. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific troponin. Variants may be allelic variants or any other species specific homologs, paralogs, or orthologs. Moreover, the variants referred to herein include fragments of the specific cardiac troponins or the aforementioned types of variants as long as these fragments have the essential immunological and biological properties as referred to above. Such fragments may be, e.g., degradation products of the troponins. Further included are variants which differ due to posttranslational modifications such as phosphorylation or myristylation.

CRP, herein also referred to as C-reactive protein, is an acute phase protein that was discovered more than 75 years ago to be a blood protein that binds to the C-polysaccharide of pneumococci. CRP is known as a reactive inflammatory marker and is produced by a distal organ (i.e., the liver) in response or reaction to chemokines or interleukins originating from the primary lesion site. CRP consists of five single subunits, which are non covalently linked and assembled as a cyclic pentamer with a molecular weight of approximately 110-140 kDa. The term “CRP” preferably also includes variants of CRP (for a definition of variants see herein above). Preferably, CRP as used herein relates to human CRP. The sequence of human CRP is well known and disclosed, e.g., by Woo et al. (J. Biol. Chem. 1985. 260 (24), 13384-13388). The level of CRP is usually low in normal individuals but can rise 100- to 200-fold or higher due to inflammation, infection or injury (Yeh (2004) Circulation. 2004; 109:II-11-II-14). It is known that CRP is an independent factor for the prediction of a cardiovascular risk. Particularly, it has been shown that CRP is suitable as a predictor for myocardial infarction, stroke, peripheral arterial disease and sudden cardiac death. Moreover, elevated CRP amounts may also predict recurrent ischemia and death in subjects with acute coronary syndrome (ACS) and those undergoing coronary intervention. Determination of CRP is recommended by expert panels (e.g., by the American Heart Association) in patients with a risk of coronary heart disease (see also Pearson et al. (2003) Markers of Inflammation and Cardiovascular Disease. Circulation, 107: 499-511).

Preferably, the amount of CRP in a sample of a subject is determined by using CRP assays with a high sensitivity. The CRP determined by such assays is frequently also referred to as high sensitivity CRP (hsCRP). HsCRP assays are, e.g., used to predict the risk of heart disease. Suitable hsCRP assays are known in the art. A particularly preferred hsCRP assay in the context of the present invention is the Roche/Hitachi CRP (Latex) HS test with a detection limit of 0.1 mg/l.

sCD40L (soluble CD40 ligand) is an 18 kDa soluble protein found in blood. sCD40L is released by proteolysis from CD40L (CD40 ligand, also known as CD154), a 33 kDa type II transmembrane protein, which belongs to the TNF superfamily of transmembrane proteins. The structure and the composition of sCD40L is well known in the art, see, e.g., Karpusas et al., Structure 1995, 3:1019-39 or Anand et al., Thromb Haemost., 90:377-84. It has been suggested that 95% of sCD40L detectable in blood originates from blood platelets (Andre, P., et al. (2002): CD40L stabilizes arterial thrombi by a B3 integrin-dependent mechanism. Nature Medicine, vol. 8, 247-52). Moreover, the term shall include variants of sCD40L. Examples of particular variants of sCD40L and methods for their measurement are known. For example, there are different variants of sCD40L (see e.g., Pietravalle, F, et al. (1996). Human Native Soluble CD40L is a Biologically Active Trimer, Processed Inside Microsomes. J Biol Chem, vol. 271, pp. 5965-7). Preferably, the term “sCD40L” relates to human sCD40L. (for an explanation of the term variant can be found herein above).

Determining the amount of the peptides or polypeptides referred to in this specification relates to measuring the amount or concentration, preferably semi-quantitatively or quantitatively. Also contemplated is the determination of variants of said peptides of and polypeptides. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e., a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g., reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on ELECSYS analyzers, Roche Diagnostics GmbH), CBA (an enzymatic cobalt binding assay, available for example on Roche-Hitachi analyzers), and latex agglutination assays (available for example on Roche-Hitachi analyzers).

Preferably, determining the amount of a peptide or polypeptide comprises the steps of (a) contacting a cell capable of eliciting a cellular response the intensity of which is indicative of the amount of the peptide or polypeptide with the peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprises the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g., phage display. Antibodies as referred to herein include both polyclonal and monoclonal antibodies, as well as fragments thereof, such as Fv, Fab and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g., according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g., by NMR or surface plasmon resonance.

Second, if the ligand also serves as a substrate of an enzymatic activity of the peptide or polypeptide of interest, an enzymatic reaction product may be measured (e.g., the amount of a protease can be measured by measuring the amount of cleaved substrate, e.g., on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable label prior to the reaction. Preferably, the sample is contacted with the substrate for an adequate period of time. An adequate period of time refers to the time necessary for a detectable, preferably measurable, amount of product to be produced. Instead of measuring the amount of product, the time necessary for appearance of a given (e.g., detectable) amount of product can be measured.

Third, the ligand may be coupled covalently or non-covalently to a label allowing detection and measurement of the ligand. Labelling may be done by direct or indirect methods. Direct labelling involves coupling of the label directly (covalently or non-covalently) to the ligand. Indirect labelling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxigenin, His-Tag, glutathione-S-transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g., magnetic beads”, including paramagnetic and superparamagnetic labels), and fluorescent labels. Enzymatically active labels include e.g., horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star (Amersham Biosciences), ECF (Amersham Biosciences). A suitable enzyme-substrate combination may result in a colored reaction product, fluorescence or chemiluminescence, which can be measured according to methods known in the art (e.g., using a light-sensitive film or a suitable camera system). As for measuring the enzymatic reaction, the criteria given above apply analogously. Typical fluorescent labels include fluorescent proteins (such as GFP and its derivatives), CY3, CY5, TEXAS RED, fluorescein, and the Alexa dyes (e.g., Alexa 568). Further fluorescent labels are available e.g., from Molecular Probes (Oregon). Also the use of quantum dots as fluorescent labels is contemplated. Typical radioactive labels include 35S, 1251, 32P, 33P and the like. A radioactive label can be detected by any method known and appropriate, e.g., a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamid gel electrophoresis (SDS-PAGE), Western Blotting, and mass spectrometry), can be used alone or in combination with labelling or other detection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form.

Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20 (1):9-12). In such suspension arrays, the carrier, e.g., a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The amounts of the various peptides and polypeptides as referred to herein (CRP, sCD40L, troponin T), are, preferably determined in the same sample, i. e. only in a single sample, of a respective subject. However, it is also envisaged by the method of the present invention that said peptides and polypeptides are determined in different samples, i.e., more than one sample of a respective subject. Most preferably, the peptides and polypeptides are determined simultaneously. However, the amount of the markers may also be determined sequentially.

The term “amount” as used herein encompasses the absolute amount of a polypeptide or peptide, the relative amount or concentration of the polypeptide or peptide as well as any value or parameter which correlates thereto or can be derived therefrom. Such values or parameters comprise intensity signal values from all specific physical or chemical properties obtained from the peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in step (b) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e., automatically provide the desired assessment in a suitable output format. Based on the comparison of the amount(s) determined in order to carry out the method of the present invention and the reference amount (s), it is possible to assess whether a subject as defined herein is susceptible to a cardiac therapy. Therefore, the reference amount is to be chosen so that either a difference or a similarity in the compared amounts allows identifying those subjects which are susceptible to a cardiac therapy.

Accordingly, the term “reference amounts” as used herein refers to amounts of the polypeptides which allows for identifying a subject susceptible to a cardiac therapy among those subjects with stable coronary heart disease and a history of cardiovascular events. Accordingly, the reference may either be derived from (i) a subject with stable coronary heart disease and a history of cardiovascular events and who is known to be susceptible to a cardiac therapy, or (ii) a subject with stable coronary heart disease and a history of cardiovascular events and who is known not to be susceptible to a cardiac therapy. Moreover, the reference amounts, preferably, define thresholds. Suitable reference amounts or threshold amounts may be determined by the method of the present invention from a reference sample to be analyzed together, i.e., simultaneously or subsequently, with the test sample. A preferred reference amount serving as a threshold may be derived from the upper limit of normal (ULN), i.e., the upper limit of the physiological amount to be found in a population of subjects (e.g., patients enrolled for a clinical trial).

Thus, the reference amount defining a threshold amount for a cardiac troponin and, in particular, for troponin T as referred to in accordance with the present invention is, preferably, 0.02 ng/ml, and, more preferably, 0.01 ng/ml and, most preferably, 0.007 ng/ml.

The reference amount defining a threshold amount for CRP as referred to in accordance with the present invention is, preferably, 6.0 μg/ml, more preferably, 4.0 μg/ml and, most preferably, 2.8 μg/ml.

Thus, the reference amount defining a threshold amount for sCD40L, as referred to in accordance with the present invention is, preferably, 3.75 μg/ml, more preferably, 2.75 μg/ml and, most preferably, 2 μg/ml.

Preferably, (i) an amount of a cardiac troponin, and, particularly, of troponin T larger than the reference amount for a cardiac troponin, and, particularly, for a cardiac troponin T, and (ii) an amount of the C-reactive protein larger than the reference amount for the C-reactive protein is indicative for a subject being highly susceptible to a cardiac therapy. Highly susceptible, preferably, means that the subject is susceptible to (and thus requires) interventional methods as described above. Particularly, care should be taken to restore blood flow by said methods as soon as possible, preferably within the next three months, two months, or, more preferably, within the next four weeks (after the sample was obtained). Of course, said subject may also take drugs as summarized above. Preferred drugs are anti-inflammatory drugs (particularly statins), ACE inhibitors, aldosterone antagonists, and Angiotensin receptor blocker.

Preferably, (i) an amount of a cardiac troponin, and, particularly, of troponin T larger than the reference amount for said cardiac troponin, and (ii) an amount of the C-reactive protein lower than the reference amount for the C-reactive protein is indicative for a subject being susceptible to a cardiac therapy. Preferably, the focus of treatment will be medication with drugs as mentioned above, preferably, with anti-inflammatory drugs such as statins and/or with lipid-lowering drugs.

Preferably, (i) an amount of a cardiac troponin, and, particularly, of troponin T lower than the reference amount for a cardiac troponin, and, particularly, for a cardiac troponin T, and (ii) an amount of sCD40L larger than the reference amount for sCD40L is indicative for a subject being susceptible to a cardiac therapy. Preferably, the focus of treatment will be medication with drugs as mentioned above, preferably, with thrombocyte aggregation inhibitors (particularly, clopidogrel, macumar, heparin and acetylsalicylic acid) and lipid-lowering drugs.

Preferably, (i) an amount of a cardiac troponin, and, particularly, of troponin T lower than the reference amount for a cardiac troponin, and, particularly, for a cardiac troponin T, and (ii) an amount of sCD40L lower than the reference amount for sCD40L is indicative for a subject not being susceptible to a cardiac therapy.

In the studies underlying the present invention, the amounts of troponin T, hsCRP, and sCD40L were determined in serum samples obtained from a total of 670 individuals with stable coronary heart disease and a history of an acute cardiovascular event (unstable angina) in the past. Advantageously, it has been found that determining the amount of a cardiac troponin, and, if the amount of said cardiac troponin is larger than a suitable reference amount for said cardiac troponin, determining the amount of CRP, or, if the amount of said cardiac troponin is lower than a suitable reference amount for said cardiac troponin, determining the amount of sCD40L, is required for identifying more reliably a subject being susceptible to a cardiac therapy. Cardiac troponins are known markers for coronary heart disease. Several studies indicated a connection between the troponin level in a subject and the severity of the heart disease. However, in the analysis done for the present invention, it was shown that the determination of further markers in addition to cardiac troponin, namely sCD40L and CRP, allows a more reliable assessment of the extent of a heart disease, and therefore, to identify subjects being susceptible to a cardiac therapy and assessing the risk of mortality and/or a further acute cardiovascular event.

Particularly, in patients with low levels of a cardiac troponin, the determination of sCD40L adds further prognostic and diagnostic value, whereas in patients with increased levels of a cardiac troponin, the determination of CRP adds further prognostic and prognostic value.

The method of the present invention, advantageously, allows for a reliable, fast and less cost intensive diagnosis and can be implemented even in portable assays, such as test strips. Therefore, the method is particularly well suited for the identification of subjects being susceptible to a cardiac therapy. Thanks to the findings of the present invention, a suitable therapy for a subject can be reliably selected, e.g., a therapy for stable coronary heart disease. Moreover, it was shown, that the determination of sCD40L in subjects with troponin levels above the reference amount, and that the determination of CRP in subjects with troponin levels below the reference, does not add further prognostic and diagnostic value.

Thus, due to the findings of the present invention, a suitable therapy for a subject can be reliably selected. Severe side effects caused by the wrong treatment of patients can be avoided. Moreover, cost can be saved.

In addition, carrying out the method of the present invention also allows assessing the severity of stable coronary heart disease (and thus is a method for subclassification of subjects as described herein) and/or allows making decisions on a treatment. Therefore, the present invention also relates to method for a assessing the severity of a stable coronary heart disease in a subject comprising the steps as described herein. Accordingly, a subject who is susceptible to a cardiac intervention also suffers from a more severe form of CHD, whereas as subject who is not susceptible to a cardiac intervention suffers from a less severe form of CHD. Also envisaged is a method for deciding on a treatment for a subject based in the steps as described herein.

The definitions given above apply mutatis mutandis to the following.

The present invention also relates to a method for predicting the risk of mortality and/or a further acute cardiovascular event for a subject with stable coronary heart disease and a history of an acute cardiovascular event, comprising the steps of
a) determining the amount of a cardiac troponin in a sample of said subject,
b) comparing the amount of a cardiac troponin determined in step a) to a reference amount for a cardiac troponin,
   b1) if the determined amount of said cardiac troponin is larger than the reference amount for a cardiac troponin, determining the amount of C-reactive protein (CRP) in a sample of said subject, and compare the determined amount of CRP to a reference amount for CRP, or
   b2) if the determined amount of said cardiac troponin is lower than the reference amount for a cardiac troponin, determining the amount of sCD40L (soluble CD40L ligand) in a sample of said subject, and compare the determined amount of sCD40L to a reference amount for sCD40L, and
c) predicting the risk of mortality and/or a further acute cardiovascular event for said subject.

The term “predicting” as used herein relates to assessing the probability according to which a subject suffering from stable coronary heart disease and having a history of at least one cardiovascular event will die (e.g., mortality caused by the heart disease) or develop a cardiovascular event, preferably an acute cardiovascular event such as an acute coronary syndrome (ACS) within a defined time window (predictive window) in the future. The predictive window is an interval in which the subject will develop a cardiovascular event or will die according to the predicted probability. The predictive window may be the entire remaining lifespan of the subject upon analysis by the method of the present invention. Preferably, however, the predictive window is an interval of one month, six months or one, two, three, four, five or ten years after carrying out the method of the present invention (more preferably and precisely, after the sample to be analyzed by the method of the present invention has been obtained). As will be understood by those skilled in the art, such an assessment is usually not intended to be correct for 100% of the subjects to be analyzed. The term, however, requires that the assessment will be valid for a statistically significant portion of the subjects to be analyzed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the prediction will be correct for at least 60%, at least 70%, at least 80%, or at least 90% of the subjects of a given cohort.

The term “mortality” as used herein relates to mortality from any cause, preferably, from a further acute cardiovascular event. Moreover, mortality can also refer to the death rate or the ratio of number of deaths to a given population of subjects. The term “acute cardiovascular event” has been defined elsewhere in this specification.

The term “predicting the risk of mortality and/or a further acute cardiovascular event” as used herein means that it the subject to be analyzed by the method of the present invention is allocated either into the group of subjects of a population having a normal, i.e., non-elevated and, thus, average risk for developing an acute cardiovascular event or mortality, or into a group of subjects having a elevated risk, or into a group of subjects having a significantly elevated risk. An elevated risk as referred to in accordance with the present invention also means that the risk of developing a cardiovascular event or the risk of mortality within a predetermined predictive window is elevated for a subject with respect to the average risk for a cardiovascular event or mortality in a population of subjects as defined herein. Preferably, for a predictive window of one year, the average risk is within the range of 2.0 and 3.0%, preferably, 2.5%. An elevated risk as used herein, preferably, relates to a risk of more than 3.0%, preferably, more than 2.5%, and, most preferably within 3.0% and 4.0%, with respect to a predictive window of one year. A significantly elevated risk as used herein, preferably relates to a risk more than 4.0%, more preferably more than 5%, and most preferably within the range of 4.0% and 8.0%, or within the range of 5.0 and 8.0 or even higher with respect to a predictive window of one year.

A reference amount may either be derived from (i) a subject with stable coronary heart disease and a history of cardiovascular events who had a further acute cardiovascular event and/or died within a defined window period, or (ii) a subject with stable coronary heart disease and a history of cardiovascular events and who had no further acute cardiovascular event and/or did not die within a defined window period. Preferred reference amounts for the various markers CRP, cardiac troponin and sCD40L are indicated herein above.

Preferably, an amount of a cardiac troponin, preferably, of troponin T larger than the reference amount for troponin, and an amount of the C-reactive protein larger than the reference amount for the C-reactive protein is indicative for a significantly elevated risk of mortality and/or a further acute cardiovascular event.

Preferably, an amount of a cardiac troponin, preferably, of troponin T larger than the reference amount for troponin and an amount of the C-reactive protein lower than the reference amount for the C-reactive protein is indicative for an elevated risk of mortality and/or a further acute cardiovascular event.

Preferably, an amount of a cardiac troponin, preferably, of troponin T lower than the reference amount for troponin, and an amount of sCD40L larger than the reference amount for sCD40L is indicative for an elevated risk of mortality and/or a further acute cardiovascular event.

Preferably, an amount of a cardiac troponin, preferably, of troponin T lower than the reference amount for troponin, and an amount of sCD40L lower than the reference amount for sCD40L is indicative for a subject not being at elevated risk (and, thus, being at average risk) of mortality and/or a further acute cardiovascular event.

Furthermore, the present invention relates to a method of identifying a subject being susceptible to a cardiac therapy comprising the steps of
a) determining the amount of CRP in a sample of a subject with stable coronary heart disease, and a history of an acute cardiovascular event, having a cardiac troponin (preferably troponin T) level of, larger than, preferably, 0.02 ng/ml, and, more preferably larger than 0.01 ng/ml and, most preferably larger than 0.007 ng/ml, (but preferably also, lower than, 0.25 ng/ml, more preferably, lower than 0.05 ng/ml, and, most preferably, lower than 0.1 ng/ml),
b) comparing the amount of CRP determined in step a) to a reference amount for CRP, and
c) identifying a subject being susceptible to a cardiac therapy.

Preferred reference amounts for CRP are disclosed herein above.

Preferably, an amount of CRP larger than the reference amount for CRP in a sample of the subject referred to in the context of the aforementioned method indicates that said subject is highly susceptible to a cardiac therapy. Preferably, an amount of CRP lower than the reference amount for CRP in a sample of a subject indicates that said subject is susceptible to cardiac therapy.

Also, the present invention relates to a method for predicting the risk of mortality and/or a further acute cardiovascular event for a subject with stable coronary heart disease and a history of an acute cardiovascular event, said subject having a cardiac troponin (preferably troponin T) level of, larger than, preferably, 0.02 ng/ml, and, more preferably, larger than 0.01 ng/ml and, most preferably, larger than 0.007 ng/ml, but also preferably, lower than, 0.25 ng/ml, more preferably, lower than 0.05 ng/ml, and, most preferably, lower than 0.1 ng/ml
a) determining the amount of CRP in a sample of said subject,
b) comparing the amount of CRP determined in step a) to a reference amount for a CRP, and
c) predicting the risk of mortality and/or a further acute cardiovascular event for said subject.
Preferred reference amounts for CRP are disclosed herein above.

Preferably, an amount of CRP larger than the reference amount for CRP in a sample of the subject as referred to in the context of the aforementioned method indicates that said subject is at significantly elevated risk of mortality and/or a further acute cardiovascular event. Preferably, an amount of CRP lower than the reference amount for CRP in a sample of a subject indicates that said subject is at elevated risk of mortality and/or a further acute cardiovascular event

Furthermore, the present invention relates to a method of identifying a subject being susceptible to a cardiac therapy comprising the steps of
a) determining the amount of sCD40L in a sample of a subject with stable coronary heart disease, with a history of an acute cardiovascular event and having a cardiac troponin (preferably troponin T) level of lower than, preferably, 0.02 ng/ml, and, more preferably, lower than 0.01 ng/ml and, most preferably, lower than 0.007 ng/ml,
b) comparing the amount of sCD40L determined in step a) to a reference amount for sCD40L, and
c) identifying a subject being susceptible to a cardiac therapy.

Preferred reference amounts for sCD40L are disclosed herein above.

Preferably, an amount of sCD40L larger than the reference amount for sCD40L in the sample of a subject as referred to in the context of the aforementioned method indicates that said subject is susceptible to a cardiac therapy. Preferably, an amount of sCD40L lower than the reference amount for sCD40L in a sample of a subject indicates that said subject is not susceptible to cardiac therapy.

Also, the present invention relates to a method for predicting the risk of mortality and/or a further acute cardiovascular event for a subject with stable coronary heart disease and a history of an acute cardiovascular event, said subject having a cardiac troponin (preferably troponin T) level of, preferably, lower than, preferably, 0.02 ng/ml, and, more preferably, 0.01 ng/ml and, most preferably, lower than 0.007 ng/ml comprising the steps of
a) determining the amount of sCD40L in a sample of said subject,
b) comparing the amount of sCD40L determined in step a) to a reference amount for sCD40L, and
c) predicting the risk of mortality and/or a further acute cardiovascular event for said subject.

Preferred reference amounts for sCD40L are disclosed herein above.

Preferably, an amount of sCD40L larger than the reference amount for sCD40L in a sample of a subject indicates that said subject is at elevated risk for mortality and/or a further acute cardiovascular event. Preferably, an amount of sCD40L lower than the reference amount for sCD40L in a sample is at average risk of mortality and/or of a further acute cardiovascular event (and, thus, not being at elevated risk)

Furthermore the present invention relates to a device for identifying a subject being susceptible to a cardiac therapy comprising (a) means for determining the amount of a cardiac troponin in a sample of a subject with stable coronary heart disease and a history of an acute cardiovascular event, and means for comparing the amount determined by said means to a reference amount for a cardiac troponin, and (b) means for determining the amount of CRP and/or sCD40L in a sample of a said subject, and means for comparing the amount determined by said means to a reference amount for CRP and/or sCD40L, whereby a subject being susceptible to a cardiac therapy is identified.

Moreover, the present invention relates to a device for predicting the risk of mortality and/or a further acute cardiovascular event in a subject with stable coronary heart disease and a history of an acute cardiovascular event comprising (a) means for determining the amount of a cardiac troponin in a sample of said subject, and means for comparing the amount determined by said means to a reference amount for a cardiac troponin, and (b) means for determining the amount of CRP and/or sCD40L in a sample of a said subject, and means for comparing the amount determined by said means to a reference amount for CRP and/or sCD40L, whereby the risk of mortality and/or a further acute cardiovascular event is predicted.

Also contemplated by the present invention is a device for identifying a subject being susceptible to a cardiac therapy and/or a device for predicting the risk of mortality and/or a further acute cardiovascular event for a subject, wherein said subject has a cardiac troponin level of larger than, preferably, 0.02 ng/ml, and, more preferably larger than 0.01 ng/ml and, most preferably, larger than 0.007 ng/ml, but also preferably, lower than, 0.25 ng/ml, more preferably, lower than 0.05 ng/ml, and, most preferably, lower than 0.1 ng/ml, said subject having stable coronary heart disease and a history of an acute cardiovascular event, said device comprising (a) means for determining the amount of CRP in a sample of a said subject, and means for comparing the amount determined by said means to a reference amount for CRP, whereby a subject being susceptible to a cardiac therapy is identified or whereby the risk of mortality and/or a further acute cardiovascular event is predicted.

Further envisaged by the present invention is a device for identifying a subject being susceptible to a cardiac therapy and/or a device for predicting the risk of mortality and/or a further acute cardiovascular event in a subject, said subject having a cardiac troponin (preferably troponin T) level of lower than, preferably, 0.02 ng/ml, and, more preferably, lower than 0.01 ng/ml and, most preferably, lower than 0.007 ng/ml, said subject also having stable coronary heart disease and a history of an acute cardiovascular event, said device comprising a) means for determining the amount of sCD40L in a sample of a said subject, and means for comparing the amount determined by said means to a reference amount for sCD40L, whereby a subject being susceptible to a cardiac therapy is identified and/or whereby the risk of mortality and/or a further acute cardiovascular event is predicted.

The term “device” as used herein relates to a system of means comprising at least the aforementioned means operatively linked to each other as to allow the prediction. Preferred means for determining the amount of a cardiac troponin, CRP, and sCD40L and means for carrying out the comparison are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. Alternatively, where means such as test strips are used for determining the amount of the peptides or polypeptides, the means for comparison may comprise control strips or tables allocating the determined amount to a reference amount. The test strips are, preferably, coupled to a ligand which specifically binds to the peptides or polypeptides referred to herein. The strip or device, preferably, comprises means for detection of the binding of said peptides or polypeptides to the ligand. Preferred means for detection are disclosed in connection with embodiments relating to the method of the invention above. In such a case, the means are operatively linked in that the user of the system brings together the result of the determination of the amount and the prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test strips or electronic devices which merely require loading with a sample. The results may be given as output of raw data which need interpretation by the clinician. Preferably, the output of the device is, however, processed, i.e., evaluated, raw data the interpretation of which does not require a clinician. Further preferred devices comprise the analyzing units/devices (e.g., biosensors, arrays, solid supports coupled to ligands specifically recognizing the biomarkers, Plasmon surface resonace devices, NMR spectrometers, mass-spectrometers etc.) or evaluation units/devices referred to above in accordance with the method of the invention.

Further envisaged by the present invention is a kit adapted to carry out the method of the present invention for identifying a subject being susceptible to a cardiac therapy, said kit comprising instructions for carrying out the method and (a)means for determining the amount of a cardiac troponin in a sample of a subject with stable coronary heart disease and a history of at least one acute cardiovascular event, and means for comparing the amount determined by said means to a reference amount for a cardiac troponin, and (b) means for determining the amount of CRP and/or sCD40L in a sample of a said subject, and means for comparing the amount determined by said means to a reference amount for CRP and/or sCD40L, allowing identifying a subject being susceptible to a cardiac therapy.

Also contemplated by the present invention is a kit adapted to carry out the method of the present invention for predicting the risk of mortality and/or a further acute cardiovascular event in a subject, said kit comprising instructions for carrying out the method and (a) means for determining the amount of a cardiac troponin in a sample of a subject with stable coronary heart disease and a history of at least one acute cardiovascular event, and means for comparing the amount determined by said means to a reference amount for a cardiac troponin, and (b) means for determining the amount of CRP and/or sCD40L in a sample of a said subject, and means for comparing the amount determined by said means to a reference amount for CRP and/or sCD40L, allowing prediction of the risk of mortality and/or a further acute cardiovascular event.

Further envisaged by the present invention is a kit adapted to carry out the method of the present invention for identifying a subject being susceptible to a cardiac therapy and/or a kit for predicting the risk of mortality and/or a further acute cardiovascular event in a subject, said subject having a cardiac troponin level of larger than, preferably, 0.02 ng/ml, and, more preferably larger than 0.01 ng/ml and, most preferably, larger than 0.007 ng/ml, but also preferably, lower than, 0.25 ng/ml, more preferably, lower than 0.05 ng/ml, and, most preferably, lower than 0.1 ng/ml, and having stable coronary heart disease and a history of an acute cardiovascular event, said kit comprising instructions for carrying out the method and (a) means for determining the amount of CRP in a sample of a said subject, and means for comparing the amount determined by said means to a reference amount for CRP, whereby a subject being susceptible to a cardiac therapy is identified and/or whereby the risk of mortality and/or a further acute cardiovascular event is predicted.

Further envisaged by the present invention is a kit adapted to carry out the method of the present invention for identifying a subject being susceptible to a cardiac therapy and a kit for predicting the risk of mortality and/or a further acute cardiovascular event in a subject, said subject having a cardiac troponin (preferably troponin T) level of lower than, preferably, 0.02 ng/ml, and, more preferably, lower than 0.01 ng/ml and, most preferably, lower than 0.007 ng/ml, said subject also having stable coronary heart disease and a history of an acute cardiovascular event, said kit comprising instructions for carrying out the method and (a) means for determining the amount of sCD40L in a sample of a said subject, and means for comparing the amount determined by said means to a reference amount for sCD40L, whereby a subject being susceptible to a cardiac therapy is identified or whereby the risk of mortality and/or a further acute cardiovascular event is predicted.

The term “kit” as used herein refers to a collection of the aforementioned means, preferably, provided separately or within a single container. The components of the kit may be comprised by separate vials (i.e., as a kit of separate parts) or provided in a single vial. Moreover, it is to be understood that the kit of the present invention is to be used for practising the methods referred to herein above. It is, preferably, envisaged that all components are provided in a ready-to-use manner for practising the methods referred to above. Further, the kit preferably contains instructions for carrying out the methods. The instructions can be provided by a user's manual in paper- or electronic form. For example, the manual may comprise instructions for interpreting the results obtained when carrying out the aforementioned methods using the kit of the present invention.

Moreover, the present invention relates to the use of a cardiac troponin, sCD40L, and/or CRP for identifying a subject being susceptible to a cardiac therapy and to the use of a cardiac troponin, sCD40L, and/or CRP for predicting the risk of mortality and/or a further cardiovascular event in a subject as described herein.

The following examples shall merely illustrate the invention. They shall not be construed, whatsoever, to limit the scope of the invention.

### EXAMPLE 1

The amounts of troponin T, hsCRP, and sCD40L were determined in serum samples obtained from a total of 670 individuals with coronary heart disease and a history of unstable angina in the past. sCD40L was determined by use of an immunoassay for the ELECSYS 2010 automated analyzer (Roche Diagnostics GmbH, Germany). troponin T was assayed by the sensitive troponin T test from Roche Diagnostics. hsCRP was determined with the Tina-quant C-reactive protein (latex) high sensitive assay from Roche/Hitachi.

It was shown that the analyzed biomarkers are independent predictors for survival within a window period of eight years.

Moreover, in patients with troponin T levels lower than the median (6.9 pg/ml), CRP allows for differentiating between an elevated risk and a significantly elevated risk of mortality. Patient having levels of troponin and CRP above the median value, had a significantly elevated risk of mortality and a further acute cardiovascular event, whereas patients with a troponin T level above the median for troponin T, but a CRP lower than the median for CRP had an elevated risk (median CRP 2.8 mg/l).

Interestingly, in patients with troponin T levels above the median (6.9 pg/ml), sCD40L allows to differentiate between elevated risk/not elevated risk of mortality. Patient having a level of troponin lower than the median value, but having a sCD40L level larger than the median for sCD40L, had an elevated risk of mortality and a further acute cardiovascular event, whereas patients with a troponin T level and a sCD40L level lower than the respective medians of troponin T and sCD40L (median sCD40L 2 mg/l) were not at elevated risk risk.

Therefore, the use of sCD40L as a marker in subjects with low troponin T amounts allows for identifying subjects at elevated risk, and the use of CRP in subjects with elevated troponin T amounts allows for identifying subjects at significantly increased risk of mortality. Accordingly, a suitable treatment can be initiated.

### EXAMPLE 2

A 56 years old patient with known stable coronary artery disease who had an acute coronary syndrome one year ago presents at his general practitioner. troponin T (4 pg/ml) and CD401 (3.2 mg/l) are determined in a serum sample obtained from the patient. After 6 months, the patient shows up at the Emergency Room with symptoms of ACS and a NSTEMI is diagnosed (troponin T 0.2 ng/ml). After the patient has recovered from the acute event, a coronary angiography is carried out and an acute plaque with a fresh thrombus is detected

### EXAMPLE 3

A 52 years old male patient who has been diagnosed with ACS five years ago undergoes a routine examination in which troponin T (3 pg/ml) and CD401 (1.3 mg/l) are determined. An echocardiography is carried out and no abnormalities are found. Moreover, a stress test indicates (no pathological findings) that the patient is in a stable condition. As a consequence, there is no need to carry out a coronary angiography and treatment with ASS is continued. Within the following four years, there are no further acute events.

### EXAMPLE 4

A 62 years old male patient who had a NSTEMI nine month ago has a serum troponin T level of 12 pg/ml and a serum CRP level of 3.4 mg/l (without any indication of an acute infection). A therapy with ASS, statins and ACE inhibitors is continued. Seven months later, however, the patient has a myocardial infarction.

### EXAMPLE 5

A 61 years old female patient with known stable coronary artery disease had an ACS 1.5 and 3.5 years ago. The patient undergoes a routine examination in which troponin T (9.5 pg/ml) and CRP (1.6 mg/1) are determined. A stress EKG is carried out (without any abnormalities). Moreover an echocardiography is carried out (no wall motion abnormalities). Therefore, no coronary angiography is carried out and a therapy with statins, ACE inhibitors and ASS is continued. Within the following three years, there are no further acute events.

What is claimed is:

1. A method for identifying susceptibility of a subject to a cardiac therapy, wherein the subject suffers from stable coronary heart disease and has a history of at least one acute cardiovascular event, the method comprising the steps of:
determining an amount of a cardiac troponin in a sample from the subject,
comparing the amount of the cardiac troponin determined to a reference amount of the cardiac troponin, and
if the determined amount of the cardiac troponin is larger than the reference amount of the cardiac troponin, then also determining an amount of C-reactive protein (CRP) in a sample from the subject and comparing the determined amount of CRP to a reference amount of CRP, or if the determined amount of the cardiac troponin is lower than the reference amount of the cardiac troponin, then also determining an amount of soluble CD40L ligand (sCD40L) in a sample from the subject and comparing the determined amount of sCD40L to a reference amount of sCD40L, wherein
an amount of the cardiac troponin larger than the reference amount of the cardiac troponin and an amount of the C-reactive protein larger than the reference amount of the C-reactive protein is indicative for high susceptibility of the subject to the cardiac therapy,
an amount of the cardiac troponin larger than the reference amount of the cardiac troponin and an amount of the C-reactive protein lower than the reference amount of the C-reactive protein is indicative for susceptibility of the subject to the cardiac therapy,
an amount of the cardiac troponin lower than the reference amount of the cardiac troponin and an amount of sCD40L larger than the reference amount of sCD40L is indicative for susceptibility of the subject to the cardiac therapy, and
an amount of the cardiac troponin lower than the reference amount of the cardiac troponin and an amount of sCD40L lower than the reference amount of sCD40L is indicative for non-susceptibility of the subject to the cardiac therapy.

2. The method of claim 1, wherein the cardiac troponin is troponin T.

3. The method of claim 1, wherein the cardiac therapy is a drug-based therapy or an interventional therapy.

4. The method of claim 1, wherein the at least one acute cardiovascular event occurred more than six months ago.

5. The method of claim 1, wherein the sample is a blood, blood plasma, or a blood serum sample.

6. The method of claim 1, wherein the reference amount of the cardiac troponin is 0.007 ng/ml and the cardiac troponin is troponin T, the reference amount of C-reactive protein is 2.8 μg/ml, and the reference amount of sCD40L is 2.0 μg/ml.

7. A method for predicting a risk of mortality or a further acute cardiovascular event in a subject, wherein the subject suffers from stable coronary heart disease and has a history of at least one acute cardiovascular event, the method comprising the steps of:
determining an amount of a cardiac troponin in a sample from the subject,
comparing the amount of the cardiac troponin determined to a reference amount of the cardiac troponin, and
if the determined amount of the cardiac troponin is larger than the reference amount of the cardiac troponin, then also determining an amount of C-reactive protein (CRP) in a sample from the subject and comparing the determined amount of CRP to a reference amount of CRP, or if the determined amount of the cardiac troponin is lower than the reference amount of the cardiac troponin, then also determining an amount of soluble CD40L ligand (sCD40L) in a sample from the subject and comparing the determined amount of sCD40L to a reference amount of sCD40L, wherein
an amount of the cardiac troponin larger than the reference amount of the cardiac troponin and an amount of the C-reactive protein larger than the reference amount of the C-reactive protein is indicative for a significantly elevated risk of mortality or further acute cardiovascular event in the subject,
an amount of the cardiac troponin larger than the reference amount of the cardiac troponin and an amount of the C-reactive protein lower than the reference amount of the C-reactive protein is indicative for an elevated risk of mortality or further acute cardiovascular event in the subject,
an amount of the cardiac troponin lower than the reference amount of the cardiac troponin and an amount of sCD40L larger than the reference amount of sCD40L is indicative for an elevated risk of mortality or further acute cardiovascular event in the subject, and
an amount of the cardiac troponin lower than the reference amount of the cardiac troponin and an amount of sCD40L lower than the reference amount of sCD40L is indicative for no increased risk of mortality or further acute cardiovascular event in the subject.

8. The method of claim 7, wherein the reference amount of the cardiac troponin is 0.007 ng/ml and the cardiac troponin is troponin T, the reference amount of C-reactive protein is 2.8 μg/ml, and the reference amount of sCD40L is 2.0 pμg/ml.

9. A method for identifying susceptibility of a subject to a cardiac therapy, wherein the subject has stable coronary heart disease, a history of at least one acute cardiovascular event, and a cardiac troponin level of lower than 0.007 ng/ml, the method comprising the steps of:
determining an amount of C-reactive protein (CRP) in a sample from a subject,
comparing the amount of CRP determined to a reference amount of CRP, and
identifying susceptibility of the subject to the cardiac therapy if the amount of CRP determined is larger than the reference amount of CRP.

10. A method for identifying susceptibility of a subject to a cardiac therapy, wherein the subject has stable coronary heart disease, a history of at least one acute cardiovascular event, and a cardiac troponin level of lower than 0.007 ng/ml, the method comprising the steps of:
determining an amount of soluble CD40L ligand (sCD40L) in a sample from a subject,
comparing the amount of sCD40L determined to a reference amount of sCD40L, and
identifying susceptibility of the subject to the cardiac therapy if the amount of sCD40L determined is larger than the reference amount of sCD40L.

11. A device for identifying susceptibility of a subject to a cardiac therapy according to the method of claim 1, the device comprising:
means for determining an amount of a cardiac troponin in a sample from the subject, and means for comparing the amount determined with a reference amount of the cardiac troponin,
means for determining an amount of CRP in a sample from the subject and means for comparing the amount determined to a reference amount of CRP, and
means for determining an amount of sCD40L in a sample from the subject and means for comparing the amount determined to a reference amount of sCD40L,
whereby susceptibility of a subject to a cardiac therapy is identified.

12. A device for predicting a risk of mortality or a further acute cardiovascular event in a subject according to the method of claim 7, the device comprising:
means for determining an amount of a cardiac troponin in a sample from the subject, and means for comparing the amount determined with a reference amount of the cardiac troponin,
means for determining an amount of CRP in a sample from the subject and means for comparing the amount determined to a reference amount of CRP, and
means for determining an amount of sCD40L in a sample from the subject and means for comparing the amount determined to a reference amount of sCD40L,
whereby a prediction of a risk of mortality or further acute cardiovascular event is made.

13. A kit adapted for identifying susceptibility of a subject to a cardiac therapy according to the method of claim 1, the kit comprising:
instructions for carrying out the method,
means for determining an amount of a cardiac troponin in a sample from the subject,
means for determining an amount of CRP in a sample from the subject, and
means for determining an amount of sCD40L in a sample from the subject.

14. A kit adapted for predicting a risk of mortality or a further acute cardiovascular event in a subject according to the method of claim 7, the kit comprising:
instructions for carrying out the method,
means for determining an amount of a cardiac troponin in a sample from the subject,
means for determining an amount of CRP in a sample from the subject, and
means for determining an amount of sCD40L in a sample from the subject.
